(19) United States
(12) Patent Application Publication
(10) Pub. No.: US 2012/0071776 A1
(43) Pub. Date: Mar. 22, 2012

(54) HEART RATE MONITOR DEVICE AND SYSTEM

(76) Inventors: W. Wilson Keithline, New Britain, CT (US); Justin J. Morgenthau, Vernon, CT (US)

(21) Appl. No.: 12/885,008

(22) Filed: Sep. 17, 2010

(51) Int. Cl. A61B 5/044; A61B 5/04

(52) U.S. Cl. 600/523; 600/508

(57) ABSTRACT

A heart rate monitor system comprising: a user attachment means; a heart rate monitor attached to the user attachment means, and configurable to monitor the heart rate of a user during physical activity and during rest, the heart rate monitor comprising: a wireless transmitter configurable to wirelessly transmit heart rate data to a nearby receiver; a receiver, the receiver configurable to receive wireless transmissions from the wireless transmitter, and further configured to removeably connect to a portable computer device, the receiver comprising: a housing; an circuit located within the housing; a bandpass filter in signal communication with the circuit; a headset compatibility circuit in signal communication with the band pass filter; a connector means attached to the housing, and in signal communication with the headset compatibility circuit; and a portable computer device with a headset connector. A heart rate monitor receiver comprising: a receiver, the receiver configurable to receive wireless transmissions from a heart rate monitor, and further configured to removeably connect to a portable computer device, the receiver comprising: a housing; an circuit located within the housing; a bandpass filter in signal communication with the circuit; a headset compatibility circuit in signal communication with the band pass filter; a connector means attached to the housing, and in signal communication with the headset compatibility circuit.

### TECHNICAL FIELD

The present invention relates generally to heart rate monitors and more particularly to the processing of heart rate information in a portable computer device including monitoring by a heart rate monitor and wirelessly transferring the heart rate information between the heart rate monitor and the portable computer device.

### BACKGROUND

A person's heart rate during exercise is measured with a heart rate monitor (HRM) in terms of the number of heartbeats that occur during a unit of time e.g., beats per minute. The HRM may include a chest unit attached to a wearable chest strap, although other attachment means are known. The chest unit detects an electrical signal generated by the heart and displays the measured heart rate on a display unit. The display unit can additionally display indicators, such as predetermined heart rate limits or target zones.

The popularity and familiarity of the general public with portable, handheld, or pocket portable computer devices such as Blackberrys, iPhones, smart phones, MP3 players, etc. (all of which are referenced herein as a “PCD”) and other related devices has and continues to grow. However, conventional methods of transferring information to and from HRMs have been limited to communication which are not typically supported by PCDs. Currently available heart rate chest units are built with 3 different wireless communication methods: 1. 5 KHz standard analog transmission (estimated at about 47% of market); 2. 5 KHz coded analog transmission (estimated at about 47% of market); and 3. Digital proprietary transmission (estimated at about 6% of the market). There are no known devices that are interoperable with all three of the communication methods, or even with two of the three communication methods (methods 1 and 2 which comprise approximate 94% of the market).

Thus there is a need for a device and system for heart rate monitoring overcomes the above listed and other disadvantages.

### SUMMARY OF THE INVENTION

The disclosed invention relates to a heart rate monitor system comprising: a user attachment means; a heart rate monitor attached to the user attachment means, and configurable to monitor the heart rate of a user during physical activity and during rest, the heart rate monitor comprising: a wireless transmitter configurable to wirelessly transmit heart rate data to a nearby receiver; a receiver, the receiver configurable to receive wireless transmissions from the wireless transmitter, and further configured to removeably connect to a portable computer device, the receiver comprising: a housing; a circuit located within the housing; a bandpass filter in signal communication with the circuit; a headset compatibility circuit in signal communication with the band pass filter; a connector means attached to the housing, and in signal communication with the headset compatibility circuit; and a portable computer device with a headset connector.

The disclosed invention also relates to a heart rate monitor receiver comprising: a receiver, the receiver configurable to receive wireless transmissions from a heart rate monitor, and further configured to removeably connect to a portable computer device, the receiver comprising: a housing; an circuit located within the housing; a bandpass filter in signal communication with the circuit; a headset compatibility circuit in signal communication with the band pass filter; a connector means attached to the housing, and in signal communication with the headset compatibility circuit.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 is a view of the disclosed system being used by a user;

FIG. 2 is a perspective view of the disclosed receiver;

FIG. 3 is a perspective top view of the disclosed receiver from FIG. 2;

FIG. 4 is a view of the disclosed receiver about to be plugged into a PCD;

FIG. 5 is a schematic view of the disclosed system;

FIG. 6 is schematic view of the disclosed receiver and PCD showing the pass through concept; and

FIG. 7 is a flowchart illustrating a method performed by an application on the PCD.

### DETAILED DESCRIPTION

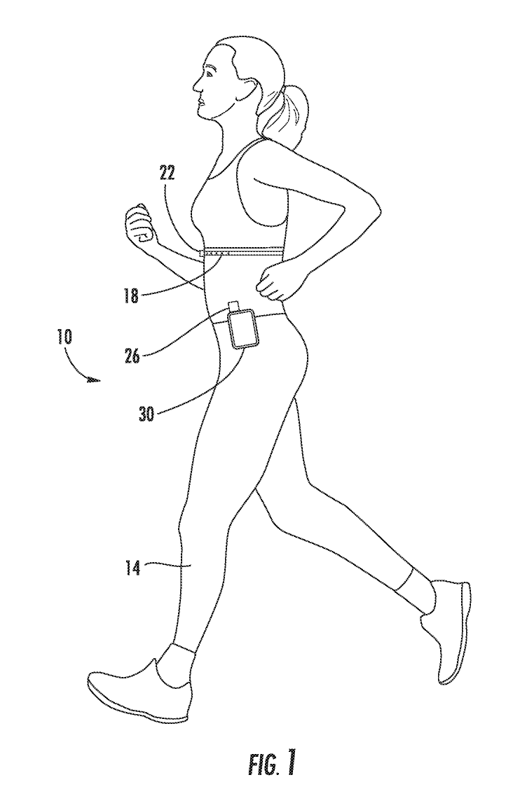

FIG. 1 shows a side view of the system 10 being used by a user 14. The system 10 comprises a user attachment means 18 with a HRM 22 attached to it, and arranged to monitor the heart rate of the user 14. In one embodiment the user attachment means 18 may be a chest strap, but other attachments means are included in the scope of this application, including but not limited to armbands, sticky pads, etc. The HRM 22 is in signal communication with a receiver 26 that is in signal communication with a PCD 30. The HRM 22 may be configured to be in wireless signal communication with the receiver 26. The receiver 26 is configured to plug into the earphone/headset plug of the PCD 30.

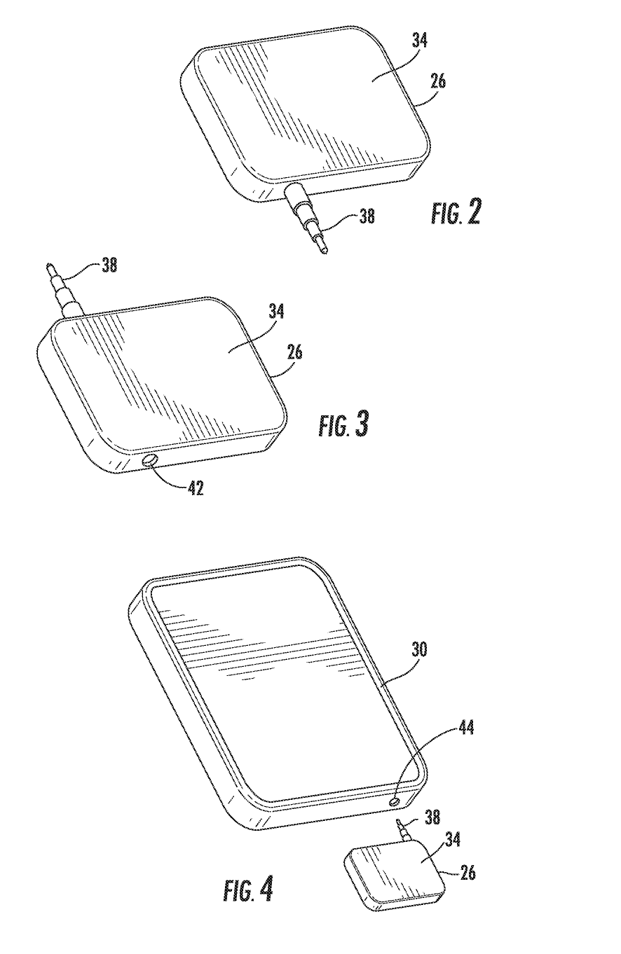

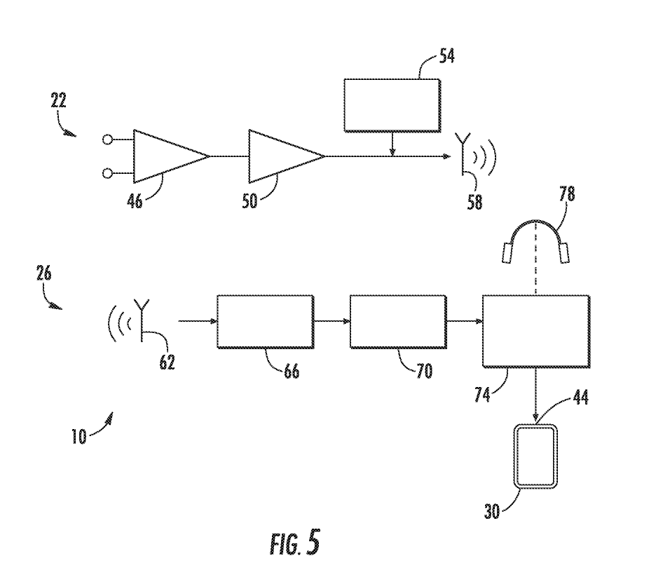

FIG. 2 shows a front perspective view of the receiver 26. The receiver comprises a housing 34 and a connector means 38. In one embodiment, the connector means 38 may be a male plug. FIG. 3 shows a front perspective view of the receiver 26 from FIG. 2, but viewed from the top of the receiver 26. In this view, a connector means 42 is shown. In one embodiment, the connector means 42 may be a female plug. FIG. 4 shows the receiver 26 about to be plugged into a PCD 30. In one embodiment, the male plug connector means 38 of the receiver plugs into the headset/earphone female connector means 44 in the PCD.

FIG. 5 shows a schematic view of the disclosed system 10. The HRM 22 is shown comprising an electrocardiogram sensor 46, and amplifier 50, an RF modulator 54, and an antenna 58. The RF modulator may have a 5.3 kHz resonance. The antenna 58 transmits a signal that is picked up by a receiver 26 antenna 62. The receiver antenna is in signal communication with a receiver circuit 66. The circuit 66 may be the discrete parts of a circuit, or an integrated circuit, or an application specific integrated circuit (ASIC). The circuit 66 is in signal communication with a band pass filter 70. Please note that the receiver does not have a CPU. This allows for the receiver 26 to be easily and inexpensively manufactured and helps keep the costs of the receiver 26 relatively low. In one embodiment the band pass filter will pass frequencies that are in the about 5 KHz range. The antenna may pickup signals from frequencies higher and lower than the desired 5 KHz frequency band. The band pass will work to filter out those signals that are higher and lower than the interested band. The band pass filter 70 is in signal communication with a headset compatibility circuit 74. The headset compatibility circuit 74 is in signal communication with a PCD 30. The receiver 26 may plug into a PCD 30 via a plug that is similar or exactly the same as a headset/mic plug. In one embodiment the head phone compatibility circuit 74 prevents the PCD 30 from detecting the headset plug and changing the PCD configuration to a configuration where a headset/mic is plugged into the PCD. In other words, even though there is a plug plugged into the headset connector 42, the PCD will not act as if a headset/mic is plugged into it. On the other hand, if a headset/mic 78 is plugged into the receiver 26 at connector 42, the headset compatibility circuit 74 will cause the PCD to recognize that a headset/mic 78 is plugged into the PCD, albeit indirectly. In another embodiment, the receiver 26 uses the microphone portion of the PCD headphone jack during heart rate operations. Should the user wish to utilize the microphone (e.g. receiving a phone call), the receiver 26 has the ability to shut-off heart rate operations and initiate microphone functions. This transition can be done manually or automatically depending on the function of the application.

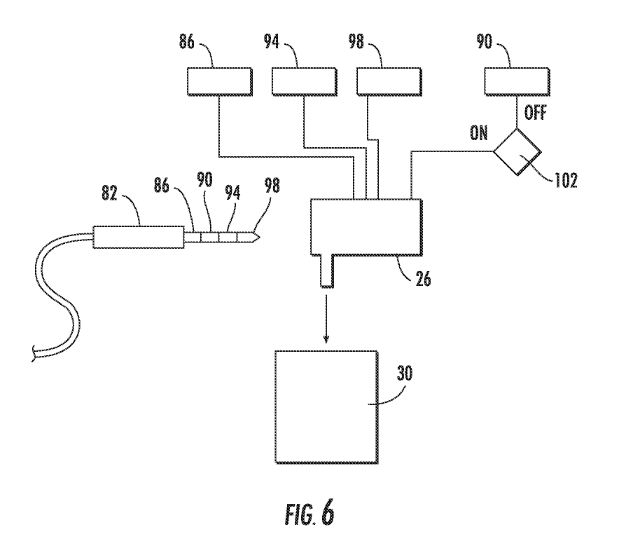

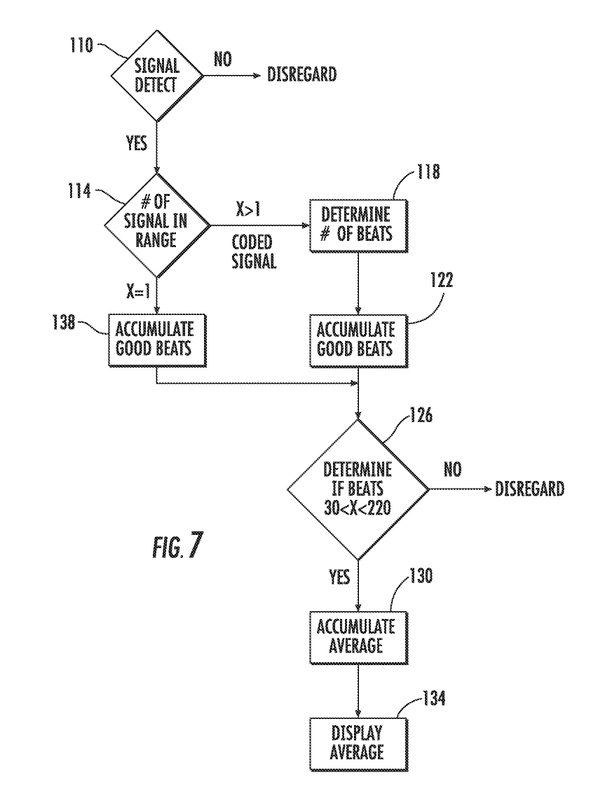

FIG. 6 shows an embodiment of the pass-through concept for a microphone. If a user wishes to use a headset 78 with a microphone along with the receiver 26, the receiver is configured to not be able to receive heart rate data from the HRM 22 and transmit to the PCD 30, when the microphone is in use. However, if a headset 78 is being used, without a microphone, then the receiver is configured to be able to receive heart rate data from the HRM 22 and transmit to the PCD 30. This feature allows one to use the HRM 22, and then switch off the receiver to allow one to use the phone function on the PCD. Thus, FIG. 6 shows a headset plug 82, the plug comprises a ground channel 86, a microphone channel 90, a left channel 94 and a right channel 98. The channels 86, 90, 94, 98 are also represented in the schematic portion of FIG. 6 in communication with the receiver 26. The receiver is in communication with a PCD 30. A switch 102 may be switched to a first position when the user is going to use the microphone, when the switch is at the first position the switch 102 deactivates the receiver 26. When the switch is turned to a second position, the switch keeps the receiver 26 activated. In another embodiment, the switch 102 may be an “auto-detect” switch. Thus, in this auto-detect embodiment, the switch 102 detects whether a microphone channel 90 is sending a signal. If a microphone signal is detected, the switch 102 deactivates the receiver 26. If a microphone signal is not detected, the switch keeps the receiver 26 activated.

FIG. 7 shows a flowchart that illustrates steps comprising the logic of an application downloaded or otherwise installed on the PCD 30. At act 110 the application determines whether a signal is detected being received by the antenna 62. At act 114 the application determines the number of signals in the signal range. The range may be a short period of time, such as about 1/10th of a second. But this can vary. So 3 signals within a 1/10th of a second period, could indicate that a coded signal is being received. If the number of signals is greater than 1, then the process moves to act 118 where the application determines the number of heart beats in the signal. At act 122, the application accumulates the number of good beats. Good beats are signals which meet certain specified criteria such as but not limited to signal amplitude and signal duration. At act 126, the application determines if the number of beats is between about 30 beats per minute and about 220 beats per minute. If the number of beats falls out of said range, the signal is disregarded. If the number of beats falls within said range, then at act 130 the application accumulates the average number of heartbeats per minute (or any other suitable time period). At act 134, the number of heartbeats per minute is displayed at the PCD 30. If at act 114, it is determined that there is one signal in range, then at act 138 the application accumulates the number of good beats.

It is contemplated that the system may, in one embodiment, operate as follows. A user wears an existing heart rate chest strap. The user plugs the disclosed wireless receiver 26 into a smart phone's headphone jack. The user starts an application on the smart phone that can interpret the heart rate signal from the disclosed receiver via the headphone jack. The application will also access other information available on the smart phone (time, gps, display, etc). The application combines this information with the heart rate information in a useful format for the user. The information can then be aggregated, transmitted to a web based application or shared via social networking applications. Ideally, multiple applications will be developed to fit the needs of the user. Categories of applications may include: bicycling, running, weight loss, sleep improvement, walking, general health, spinning, coaching, etc.

The disclosed invention has many advantages. The disclosed receiver can receive the analog signal that is compatible with both a standard and a coded analog signal, these two signal types comprise the current bulk of the market. Since the receiver also uses the headphone jack to access the smart phone, it can work with almost all smart phones which provides great interoperability and low cost. The disclosed system will allow a user to continuously upload their heart rate information onto their smart phone while the phone performs the speed and distance calculations. The system is a low cost alternative to currently available systems. The use of a PCD allows the system to display large amounts of data and in easy to use graphical forms, especially as compared to a system that uses a wristwatch style device to display the information. PCDs can upload easily to the internet. Internet applications that work with the disclosed system may store large amounts of heart rate data. The PCD may also combine GPS, location, speed and distance information along with the heart rate data. Fitness information can be aggregated with easy to use tools. Applications (or “Apps”) can be developed to focus on a large number of different market segments (weight loss, training for events, social media, etc). Heart Rate, location, speed and distance can be expressed in clear and easy to use formats. The disclosed system provides greater flexibility at a lower cost than currently known systems. Most currently known solutions require the user to perform an action to update their information onto the WEB. The disclosed system is able to update seamlessly and without action by the user. The disclosed system works with most of the existing heart rate chest straps in the market and fitness equipment. The HRM chest strap can be made compatible with all PCD devices.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A heart rate monitor system comprising:
a user attachment means;
a heart rate monitor attached to the user attachment means, and configurable to monitor the heart rate of a user during physical activity and during rest, the heart rate monitor comprising:
a wireless transmitter configurable to wirelessly transmit heart rate data to a nearby receiver;
a receiver, the receiver configurable to receive wireless transmissions from the wireless transmitter, and further configured to removeably connect to a portable computer device, the receiver comprising:
a housing;
a circuit located within the housing;
a bandpass filter in signal communication with the circuit;
a headset compatibility circuit in signal communication with the band pass filter;
a connector means attached to the housing, and in signal communication with the headset compatibility circuit; and
a portable computer device with a headset connector.

2. The heart rate monitor system of claim 1, wherein the circuit is an integrated circuit.

3. The heart rate monitor system of claim 1, wherein the user attachment means is a chest strap.

4. The heart rate monitor system of claim 1, wherein the receiver does not comprise a CPU.

5. The heart rate monitor system of claim 1, wherein the portable computer device is selected from the group consisting of blackberries, I-phones, smart phones, and portable personal computers.

6. The heart rate monitor system of claim 1, wherein the connector means is a male plug, and the headset connector is a female plug receptacle.

7. The heart rate monitor system of claim 1, further comprising:
a downloadable application installed on the portable computer device, the application configured to process and display the heart rate data from the receiver.

8. The heart rate monitor system of claim 1, further comprising a receiver switch, wherein if the switch is turned to a first position, the receiver is configured to deactivate, and if the switch is turned to a second position the receiver is configured to reactivate.

9. The heart rate monitor system of claim 1, wherein the receiver is further configured to deactivate if a microphone signal is detected, and to reactivate if a microphone signal is not detected.

10. The heart rate monitor system of claim 1, further comprising:
an application installed on the personal computer device, the application configured to perform a method that includes:
detecting a signal received by a receiver antenna;
determining the number of signals in a signal range;
accumulating good beats, if the number of signals is equal to one;
determining if the number of heartbeats is between about 30 beats per minute and 220 beats per minute;
accumulating the average number of heartbeats per minute;
displaying the average number of heartbeats per minute;
determining the number of heartbeats in the signal, if the number of signals is greater than 1;
accumulating good beats;
determining if the number of heartbeats is between about 30 beats per minute and 220 beats per minute;
accumulating the average number of heartbeats per minute; and
displaying the average number of heartbeats per minute.

11. A heart rate monitor receiver comprising:
a receiver, the receiver configurable to receive wireless transmissions from a heart rate monitor, and further configured to removeably connect to a portable computer device, the receiver comprising:
a housing;
a circuit located within the housing;
a bandpass filter in signal communication with the circuit;
a headset compatibility circuit in signal communication with the band pass filter;
a connector means attached to the housing, and in signal communication with the headset compatibility circuit.

12. The heart rate monitor receiver of claim 10, wherein the circuit is an integrated circuit.

13. The heart rate monitor receiver of claim 11, wherein the receiver does not comprise a CPU.

14. The heart rate monitor receiver of claim 11, wherein the connector means is a male plug.

15. The heart rate monitor receiver of claim 11, further comprising a receiver switch, wherein if the switch is turned to a first position, the receiver is configured to deactivate, and if the switch is turned to a second position the receiver is configured to reactivate.

16. The heart rate monitor receiver of claim 11, wherein the receiver is further configured to deactivate if a microphone signal is detected, and to reactivate if a microphone signal is not detected.
